(19) United States
(12) Patent Application Publication
(10) Pub. No.: US 2026/0314975 A1
(43) Pub. Date: Oct. 15, 2026

(54) SYSTEMS AND METHODS FOR ELECTROCARDIOGRAPHIC RADIAL DEPTH NAVIGATION OF INTRACARDIAC DEVICES

(71) Applicant: THE UNITED STATES OF AMERICA, AS REPRESENTED BY THE SECRETARY, DEPARTMENT OF HEALTH AND HUMAN, Bethesda, MD (US)

(72) Inventors: Robert J. LEDERMAN, Chevy Chase, MD (US); Christopher G. BRUCE, District of Columbia, DC (US); Dursun Korel YILDIRIM, Chevy Chase, MD (US)

(21) Appl. No.: 19/104,170

(22) PCT Filed: Aug. 14, 2023
(86) PCT No.: PCT/US2023/072161

§ 371 Date: Feb. 14, 2025

(30) Foreign Application Priority Data
Aug. 15, 2022 (US) 63371490

(51) Int. Cl. A61B 5/283; A61B 5/00; A61B 5/346

(52) U.S. Cl. CPC A61B 5/283; A61B 5/346; A61B 5/6852; A61B 5/7267

(57) ABSTRACT

Various systems and methods are provided for electrocardiographic radial depth navigation for intracardiac device insertion into a heart. In one example, a method includes acquiring an intracardiac electrogram signal via an electrode of an intracardiac device during insertion of the intracardiac device into a heart muscle or chambers and outputting a radial depth indication with respect to myocardium of the heart based on electronic processing of the intracardiac electrogram signal.

### CROSS-REFERENCE TO RELATED APPLICATIONS

The present application is a U.S. National Phase of International Application No. PCT/US2023/072161 entitled “SYSTEMS AND METHODS FOR ELECTROCARDIOGRAPHIC RADIAL DEPTH NAVIGATION OF INTRACARDIAC DEVICES”, and filed on Aug. 14, 2023. International Application No. PCT/US2023/072161 claims priority to U.S. Provisional Application No. 63/371,490, entitled “SYSTEMS AND METHODS FOR ELECTROCARDIOGRAPHIC RADIAL DEPTH NAVIGATION OF INTRACARDIAC DEVICES,” and filed Aug. 15, 2022. The entire contents of each of the above-referenced applications are hereby incorporated by reference for all purposes.

### FIELD The present description relates generally to catheter-based cardiac procedures, and electrocardiography-based methods of navigating catheter-based cardiac devices during procedures.

### BACKGROUND/SUMMARY

Some structural heart procedures use catheter-based cardiac devices, which may be navigated into a targeted position within the heart muscle via a freely steered guidewire. As one example, a procedure called myocardial intramural remodeling by transvenous tether (e.g., MIRTH) includes implanting an intra-myocardial tension element within ventricular walls to narrow the left ventricle, thereby providing a treatment for heart failure. As another example, septal scoring along the midline endocardium (e.g., SESAME) is a transcatheter myotomy procedure that may be used to relieve a left ventricular outflow tract obstruction. As yet another example, a cerclage annuloplasty is a procedure that uses a catheter to reduce mitral valve regurgitation. As yet another example, ablation treatment of ventricular arrhythmias may require localizing arrhythmia propagation pathways within the myocardium using local electrocardiography measurements from a catheter or guidewire and delivering ablative energy or agents to eliminate intra-myocardial portions of the arrhythmia propagation path.

In each of these procedures, a guidewire may be steered into the targeted position to assist implantation of the catheter-based cardiac device or to deliver ablative energy. Alternatively, the catheter-based cardiac device may be directly steered without the use of a guidewire. Various imaging techniques may be used to provide navigation feedback during guidewire/device steering, such as x-ray fluoroscopy and echocardiography. However, x-ray fluoroscopy does not enable soft tissue visualization, causing the guidewire or device to appear free-floating in space. As another example, echocardiography is hindered by off-axis imaging planes, which may cause the guidewire or device to appear deeper or more shallow than it is actually positioned. As a result, it may be difficult to determine a radial position of the guidewire or device between endocardial and epicardial surfaces of the myocardium when performing intramyocardial device navigation using traditional imaging modalities. For the purpose of this description, the term “intramyocardial” is used interchangeably with the term “intracardiac,” even though the former refers to locations within the heart muscle, and the latter refers to locations within and outside the heart muscle.

However, a radial depth of the device placement may affect procedure outcomes. For example, it may be desired to implant the intra-myocardial tension element used in the MIRTH procedure at a radial depth that is equidistant from the endocardium and the epicardium (e.g., in the mid-myocardium). As another example, it may be desired for the SESAME implant to be sufficiently deep in the myocardium to relieve left ventricular outflow obstruction but sufficiently shallow to prevent causing a ventricular septal defect. As still another example, an occurrence of heart block may be reduced by understanding a position of an exit point of the cerclage catheter. As still another example, it is desirable to navigate an ablation device within the mid-myocardium to reach an ablation target otherwise inaccessible to traditional endocardial or epicardial catheter ablation.

In one example, the issues described above may be addressed by a method that includes acquiring an intracardiac electrogram signal via an electrode of an intracardiac device (such as a guidewire or ablation catheter) during insertion of the intracardiac device into a heart muscle and incorporated chambers and outputting a radial depth indication with respect to myocardium of the heart based on electronic processing of the intracardiac electrogram signal. In this way, radial depth navigation feedback may be provided during insertion of the intracardiac device, resulting in more efficient and accurate placement of catheter-based cardiac devices, for example.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 depicts an embodiment of an electrocardiographic radial depth navigation system;

FIG. 2 shows a high-level block diagram of an electrocardiographic radial depth navigation algorithm that may be used to provide real-time radial depth navigation feedback during intracardiac device insertion into a heart;

FIG. 3 shows a block diagram of a workflow that may be used to train an intramyocardial electrogram classifier;

FIG. 4 shows example unipolar electrode data relative to myocardial radial depth;

FIG. 5 shows example waveform characteristics that may be extracted from an intracardiac electrogram signal measured by a unipolar electrode positioned at a first radial depth;

FIG. 6 shows example waveform characteristics that may be extracted from an intracardiac electrogram signal measured by a unipolar electrode positioned at a second radial depth;

FIG. 7 shows example waveform characteristics that may be extracted from an intracardiac electrogram signal measured by a unipolar electrode positioned at a third radial depth;

FIG. 8 shows example waveform characteristics that may be extracted from an intramyocardial electrogram signal measured by a unipolar electrode positioned at a fourth radial depth;

FIG. 9 shows a table summarizing qualitative features of intracardiac electrograms at different radial depth categories;

FIG. 10 shows a time-based variation in mid-myocardial electrograms from an unmoving unipolar electrode; and

FIG. 11 shows a flow-chart of an example method for providing real-time radial depth navigation feedback during intracardiac device insertion into a heart.

### DETAILED DESCRIPTION

The following description relates to systems and methods for intracardiac device navigation via electrocardiographic radial depth navigation, also referred to herein as EDEN. Examples of intracardiac devices that may be navigated via EDEN as described herein include catheters, intra-myocardial tension elements, electrophysiologic mapping and ablation catheters, myocardial biopsy devices, myocardial drug delivery catheters or needles, myocardial electrical stimulation devices, and/or myocardial pacing electrodes. In some examples, one or more of the intracardiac devices may be navigated via a guidewire, and thus a guidewire may also be an example of an intracardiac device that may be navigated via EDEN. While real-time imaging techniques such as x-ray fluoroscopy may provide information regarding a longitudinal (e.g., base-to-apex) position and a circumferential (e.g., “clock-face”) position of the intracardiac device in the heart, these techniques lack information regarding a radial position (also termed radial depth) of the intracardiac device within the myocardium. As used herein with respect to the heart, the terms “radial position” and “radial depth” denote a relative position in a single dimension between the endocardial and epicardial surfaces of the heart. As will be further described herein, the radial depth of a intracardiac device may be classified according to its relative position between the endocardial and epicardial borders (or beyond) via a depth navigation algorithm (which may be a machine learning or deep learning-based algorithm or a logic-based algorithm) that uses intramyocardial electrograms measured by an electrode of the intracardiac device itself. As such, the depth navigation algorithm may provide real-time feedback to an operator of the intracardiac device during insertion, which may increase an efficiency and accuracy of the tip position of the intracardiac device (e.g., of the guidewire).

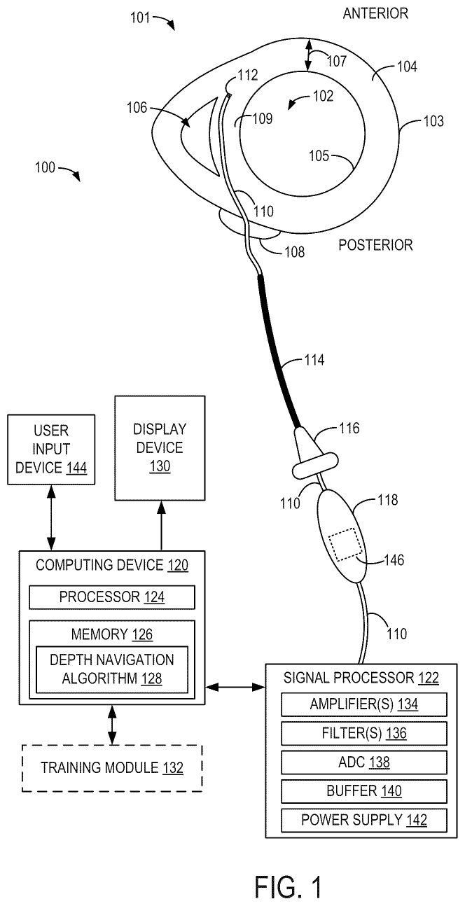

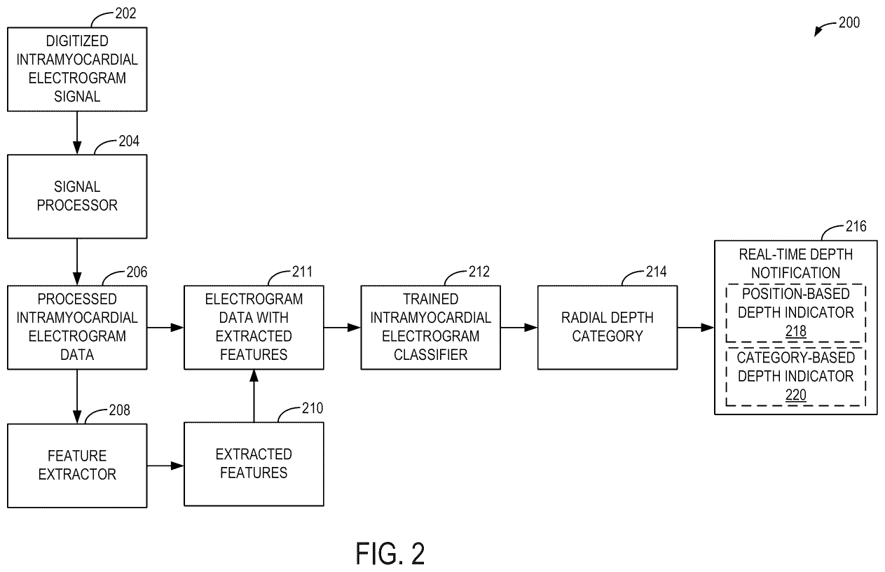

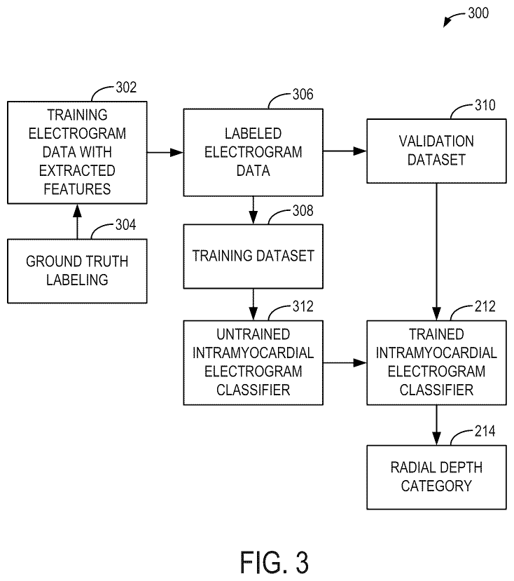

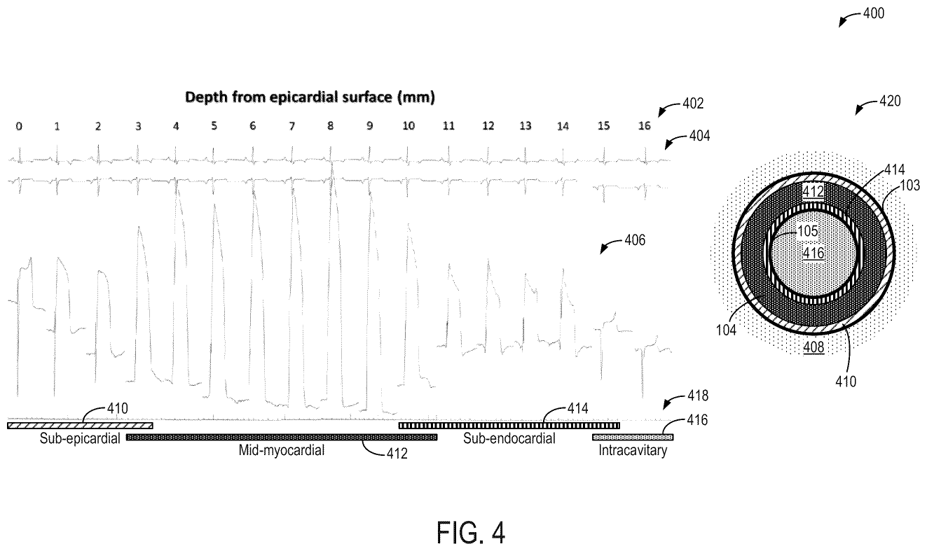

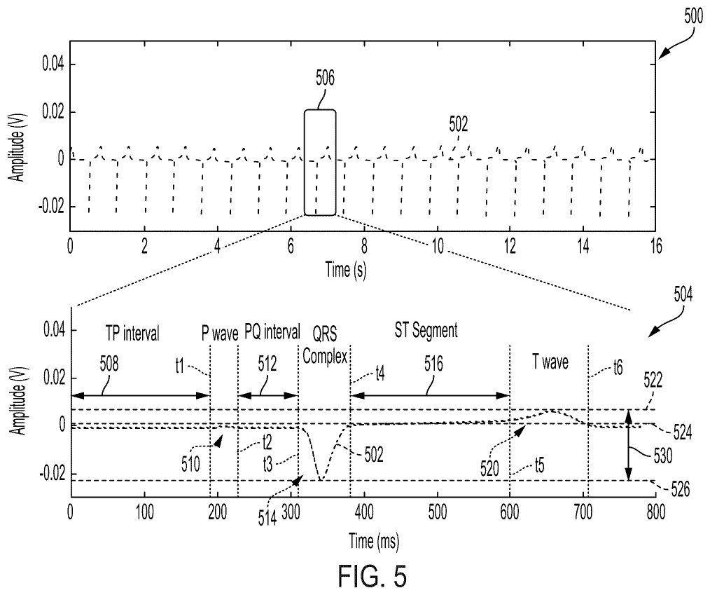

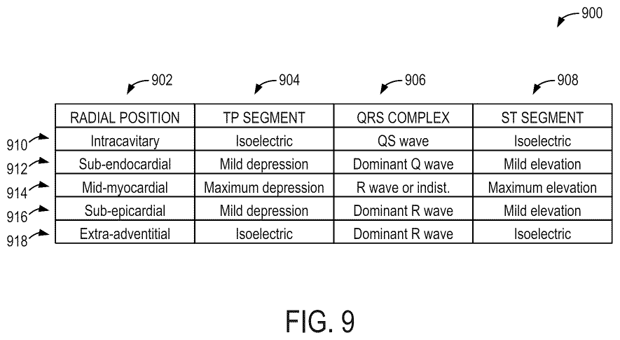

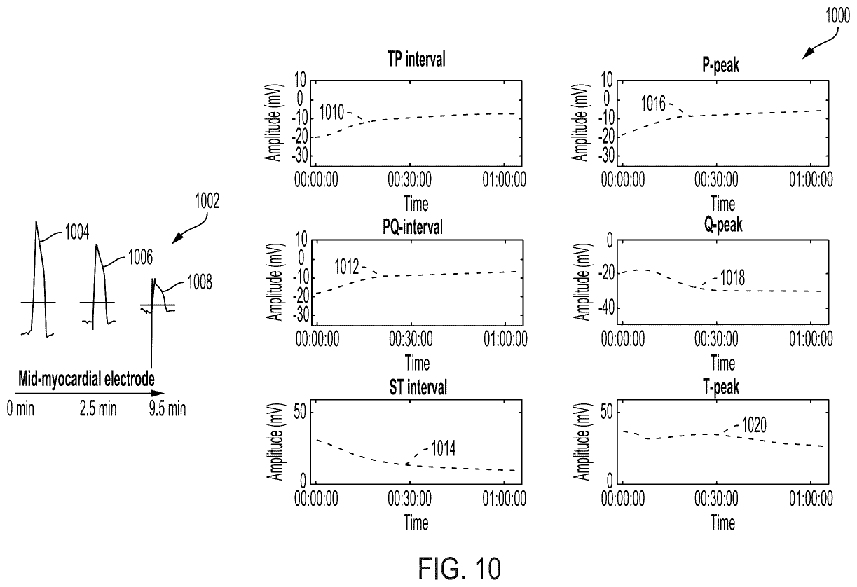

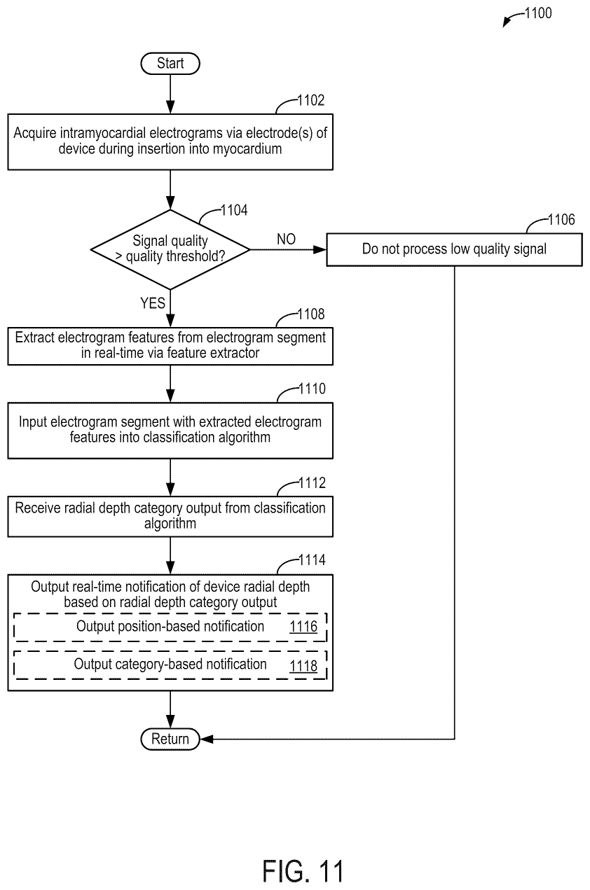

FIG. 1 shows an embodiment of an EDEN system for intramyocardial device navigation. The EDEN system may include a depth navigation algorithm, such as the depth navigation algorithm outlined in FIG. 2, for providing real-time feedback regarding a radial depth position of the intracardiac device based on electrograms acquired by an electrode of the intracardiac device. For example, the depth navigation algorithm may include a trained machine or deep learning-based classifier or logic-based classifier that may determine a radial depth category of each acquired electrogram. The classifier may be trained according to the example scheme shown in FIG. 3, for example. As demonstrated in FIG. 4, different depths between the endocardial and epicardial borders of the myocardium and outside of the myocardium produce different characteristic waveform features. These waveform features may be extracted, examples of which are shown in FIGS. 5-8, and used by the classifier to differentiate between different radial depth categories. FIG. 9 summarizes qualitative differences between waveform features at different radial depth categories. Further, the electrograms acquired by the electrode of the intracardiac device may change over time in a predictable fashion if the electrode is held in place instead of steered through the myocardium, as illustrated in FIG. 10. As such, the depth navigation algorithm may take movement of the electrode (or lack thereof) into account based on known changes to the characteristic waveform features. For example, characteristic features of consecutive electrograms can be stored in the memory. If the changes in these features are smaller than a predefined threshold, the electrode can be considered remaining in the same radial depth. Once the first condition is fulfilled, the changes in the characteristic features in time domain can be compared with the stationary electrode signal trends in FIG. 10 to confirm the stationary position of the electrode. The depth navigation algorithm may provide real-time intracardiac device navigation feedback during insertion according to the method of FIG. 11.

Turning now to the figures, FIG. 1 schematically depicts an example of an electrocardiographic radial depth navigation (EDEN) system 100 with respect to a heart 101 of a patient. The heart 101 is shown as a simplified a short-axis view depicting a cross-section of a left ventricle 102, a right ventricle 106, an interventricular septum (IVS) 109 separating the left ventricle 102 and the right ventricle 106, and a coronary sinus 108. The heart 101 includes myocardium 104, which is bordered by epicardium 103 on the exterior and endocardium 105 on the interior. As such, the myocardium 104 has a radial depth 107 from the epicardium 103 to the endocardium 105. Positions exterior to the epicardium 103 may be referred to herein as extra-adventitial or pericardial, and positions interior to the endocardium 105 may be referred to herein as intracavitary or intra-cameral.

The EDEN system 100 comprises an exposed conductor 112 positioned on a guidewire 110. The exposed conductor 112 comprises an intracardiac sensing electrode of the EDEN system 100 and may therefore also be referred to herein as an electrode. During guidewire navigation, at least a portion of the guidewire 110 is inserted into the patient, while a remaining portion of the guidewire 110 may remain external to the patient to enable guidewire manipulation and electrogram recording, as will be elaborated below. In some examples, such as the example shown, the guidewire 110 may be fed through a sheath 114 (and/or a catheter and/or microcatheter) connected to a hub 116. The sheath 114 may be a flexible material that is inserted into vasculature of the patient to provide support and controlled access for insertion of the guidewire 110. The sheath 114 may be anchored to an exterior of the patient via the hub 116, for example. The guidewire 110 may also be inserted through a torque device 118 (also called torquer), which may be used to manipulate the guidewire 110. For example, the torque device 118 may help grip and steer the guidewire 110. While FIG. 1 is described herein with respect to a guidewire having an electrode, it is to be appreciated that the EDEN system 100 may instead include another suitable intracardiac device, such as an ablation catheter, a myocardial biopsy device, a myocardial drug delivery catheter or needle, a myocardial electrical stimulation or contractility modulation device, or a myocardial pacing electrode. The intracardiac device may include one or more intracardiac sensing electrode(s) that may be used to obtain electrograms that may be processed as described herein to determine the radial depth of the intracardiac device.

The guidewire 110 comprises a thin (e.g., having an outer diameter in a range from 0.01 inches and 0.04 inches) cylindrical material having a stiffness (or flexibility) that enables insertion into and navigation within the myocardium 104. For example, the stiffness of the guidewire 110 may be greater than that used for vessel subselection procedures. Further, the tip of the guidewire 110 may be shaped to facilitate guidewire navigation by torquing and advancing. Further, the guidewire 110 may comprise an electrically conductive transmission line, such as a copper wire, that is wrapped in stainless steel and/or nickel-titanium alloy (e.g., Nitinol) braided or coiled wire for increased pushability and kink resistance. Further, a length of the guidewire 110 may be electrically insulated except for the exposed conductor 112. For example, the guidewire 110 may be coated with one or more insulators except for the exposed conductor 112 and connection points that electrically couple the exposed conductor 112 to a signal processor 122 via the electrically conductive transmission line. In this way, the guidewire 110 may include an insulated region and an uninsulated region (e.g., the exposed conductor). The electrode of the intracardiac device (e.g., the exposed conductor 112) may be a unipolar electrode in some examples. In other examples, the electrode may be bipolar or multipolar. For example, the guidewire may incorporate multiple electrically isolated insulator-conductor subassemblies for connection of multiple electrical channels to independent or multiplexed transmission line systems. In some two-conductor configurations, this would enable collection of local bipolar electrograms. The inclusion of a unipolar electrode may lower the cost and complexity of manufacturing the EDEN system 100 relative to bipolar or multipolar electrode configurations, while the bipolar or multipolar electrode configurations may generate electrograms of higher signal to noise ratio, for example.

In some examples, the guidewire 110 may be concentric with a catheter to be implanted (not shown). An outer diameter of the guidewire 110 may be less than an inner diameter of the catheter, for example, and an outer diameter of the catheter may be less than an inner diameter of the sheath 114. Once the guidewire 110 is navigated to a target location in the heart 101, the catheter may be advanced through the sheath 114 and over the guidewire 110 to position the catheter at the target position.

In some examples, the exposed conductor 112 is positioned at a distal tip of the guidewire 110, such as illustrated in FIG. 1. In other examples, the exposed conductor 112 may be positioned elsewhere along the length of the guidewire 110. Further, a length of the exposed conductor 112 may be calibrated to narrow electrogram sensing to a focal region of a few millimeters (mm) or less within the myocardium 104 while providing a large enough signal for effective acquisition. In some examples, such as the example illustrated in FIG. 1, there may be a single exposed conductor, making the exposed conductor 112 a unipolar intramyocardial electrode, while other examples may include multiple exposed conductors, as explained above. The exposed conductor 112 may be comprised of gold, stainless steel, nickel-titanium alloy, MP35N, 35N LT, silver, platinum, platinum-iridium, and/or conductive carbon, for example.

The signal processor 122 may further comprise one or more amplifiers 134, one or more filters 136, an analog-to-digital converter (ADC) 138, a buffer 140, and a power supply 142. The power supply 142 may include a battery, such as a lithium ion battery. In some examples, battery may be a rechargeable battery. Additionally or alternatively, the power supply 142 may receive power from a wall outlet (e.g., via a power adapter). The buffer 140 may be a memory component, such as a temporary storage portion of random-access memory, that stores data transferring between the signal processor 122 and other data processing components, such as a computing device 120 that will be further described below.

The one or more amplifiers 134 may comprise electrically or optically isolated amplifiers that receive and amplify an intramyocardial electrogram signal from the exposed conductor 112. The one or more filters 136 may comprise a band-pass filter(s), a band-stop filter(s), a high-pass filter(s), a low-pass filter(s), or a combination thereof. As one example, the one or more filters 136 may comprise a low-pass filter with a cut-off frequency in a range from 100 to 150 Hertz (Hz) and a high-pass filter with a cut-off frequency in a range from 0.05 to 0.5 Hz. Additionally or alternatively, a band-stop filter may be used to eliminate mains interference originating from the power supply 142 from the intramyocardial electrogram signal. For example, the band-stop filter may filter out frequencies at 50 or 60 Hz, depending on the specifications of the power supply 142.

The filtered and amplified analog intramyocardial electrogram signal may be converted to a digital signal via the ADC 138, for example, with a sampling frequency of a range between 200 to 2000 Hz. Alternatively, the intramyocardial electrogram signal may be converted to the digital signal by the ADC 138 after being amplified by the one or more amplifiers 134 and before being filtered by the one or more filters 136, such as when the one or more filters 136 are digital filters and the filtering process is performed digitally.

The EDEN system 100 further comprises the computing device 120, which may be configured to record and process the digitized electrogram signal received from the signal processor 122. The computing device 120 may be a data processor, for example. The computing device 120 may be in wired or wireless electronic communication with the signal processor 122. In other examples, the signal processor 122 may be integrated with the computing device 120, such as contained within a shared housing as a single unit. Time interval data of the digitized signal may be transferred to the computing device 120 in sliding widows. As an example, a window width of the sliding windows may be 0.2 seconds. In other examples, the window width may be adaptively adjusted based on a rhythm of the heart 101. Further, the time interval data may be stored in the buffer 140 prior to being transferred to the computing device 120 to avoid data losses. Alternatively, the signal processor 122 may transfer continuous real-time digital signal data to the computing device 120. As used herein, the term “real-time” refers to a process executed substantially at a time of occurrence. As another example, the term “real-time” refers to a process performed without intentional delay.

The computing device 120 includes a processor 124 configured to execute machine readable instructions stored in a non-transitory memory 126. The processor 124 may be single core or multi-core, and the programs executed by the processor 124 may be configured for parallel or distributed processing. In some examples, the processor 124 may optionally include individual components that are distributed throughout two or more devices, which may be remotely located and/or configured for coordinated processing. In some examples, one or more aspects of the processor 124 may be virtualized and executed by remotely-accessible networked computing devices configured in a cloud computing configuration. In some examples, the processor 124 may include other electronic components capable of carrying out processing functions, such as a digital signal processor, a FPGA, or a graphics board. In some examples, the processor 124 may include multiple electronic components capable of carrying out processing functions. For example, the processor 124 may include two or more electronic components selected from a plurality of possible electronic components, including a central processor, a digital signal processor, a field-programmable gate array, and a graphics board. In still further examples, the processor 124 may be configured as a graphical processing unit (GPU), including parallel computing architecture and parallel processing capabilities.

The non-transitory memory 126 may store the digitized intramyocardial electrogram signal received from the signal processor 122 and may further store executable programs for analyzing the digitized intramyocardial electrogram signal. In some examples, the non-transitory memory 126 may include components disposed at two or more devices, which may be remotely located and/or configured for coordinated processing. In some examples, one or more aspects of the non-transitory memory 126 may include remotely-accessible networked storage devices configured in a cloud computing configuration. In some examples, such as illustrated in FIG. 1, the non-transitory memory 126 stores a depth navigation algorithm 128. The depth navigation algorithm 128 includes one or more algorithms, including machine learning-based algorithms, deep learning-based algorithms, and/or logic-based algorithms, to process the digitized intramyocardial electrogram signal and determine the radial depth of the intracardiac device electrode (e.g., of the exposed conductor 112) in the myocardium 104 therefrom. In some examples, the depth navigation algorithm 128 may include signal processing algorithms that apply pre-programmed transformations to the digitized intramyocardial electrogram signal. For example, each time interval data segment may be vertically adjusted by aligning a mean of the signal to zero via curve fitting, such as polynomial curve fitting, to compensate for direct current (DC) bias. As one example, each data segment may be fit to a sixth order polynomial curve, although lower or high degree polynomials may be used in other examples. Further, the depth navigation algorithm 128 may apply one or more digital filters, such as a zero-phase low-pass filter, such as a sixth order Butterworth infinite impulse response (IIR) filter, to the vertically adjusted signal. The zero-phase low-pass filter may apply a cut-off frequency of 60 Hz, for example. Alternatively, lower or higher order filters may be applied. Further, other types of IIR or finite impulse response (FIR) digital filters may be applied by the depth navigation algorithm 128.

Sequentially acquired data segments of the digitized, transformed, and filtered intramyocardial electrogram signal may be concatenated and processed using sliding windows by a feature extractor of the depth navigation algorithm 128. For example, a typical surface electrocardiogram (ECG) waveform may comprise a P wave during depolarization of the atria, a QRS complex during depolarization of the ventricles, and a T wave during repolarization of the ventricles. This may result in five definable points within the waveform: P, Q, R, S, and T. The feature extractor may identify signal features, such as R-peaks and ST segment maximums, and index the detected signal features with a time stamp and/or sample number. The extracted features may further include features that describe the signal and feature extraction quality, such as signal peak, noise level, and signal-to-noise ratio (SNR). The feature extractor may further search for local extrema throughout the concatenated signal with a prominence in a range from 0.01-50. A prominence threshold may be adjusted based on the signal quality and gain of the one or more amplifiers 134. The two most recently identified consecutive signal peaks may be stored in the non-transitory memory 126. Further, the signal peak detector may be performed by measuring the slope of the time-domain signal using threshold or moving averaging techniques (e.g., the Pan and Tompkins algorithm). As another example, the signal quality may be determine based on the SNR or distinct ECG components such as the QRS complex or the ST segment. As will be elaborated below with respect to FIG. 11, a low SNR signal may be removed from further processing by the depth navigation algorithm 128.

In some examples, the depth navigation algorithm 128 may calculate the heart rate of the heart 101 using a most recent peak-to-peak interval (e.g., R-R interval). Alternatively, the heart rate may be calculated by averaging a previous number of R-R intervals. Further, the depth navigation algorithm 128 may segment a last heartbeat cycle (e.g., ECG cycle) by cutting the signal at a first percentage of the peak-to-peak interval before the signal peak (e.g., the R-peak) and at a second, remaining percentage of the peak-to-peak interval after the signal peak. For example, the first percentage may be 47%, and the second percentage may be 53%. As such, the heartbeat cycle may start at 47% of the peak-to-peak interval before the signal peak and stop at 53% of the peak-to-peak interval after the signal peak. Alternatively, the intramyocardial electrogram signal heartbeat cycle segmentation start and end points may be adjusted from the example given above, such as based on input received from an operator, such as via a user input device 144.

The feature extractor of the depth navigation algorithm 128 may identify and extract various definitive features of the intramyocardial electrogram signal, including a TP interval amplitude; a P wave amplitude; a PQ or PR interval amplitude; Q, R, and S wave amplitudes; ST segment amplitudes; a fusion of the R wave and ST segment; a range of the signal amplitude; a maximum signal amplitude; a minimum signal amplitude; structural similarities between the intramyocardial electrogram waveform and predefined waveforms; variation and standard deviation of the intramyocardial electrogram waveform; and so forth. In some examples, the intramyocardial electrogram signal may be normalized by equalizing the standard deviation of the signal to 1 and the mean of the signal to 0. Further, because ECG cycle components such as the P wave, the QRS complex, and the T wave are generated by ventricular and atrial repolarization/depolarization, there is a time-locked relationship between the components. As such, these components may be segmented using indexing the ECG segment using pre-defined time constraints. The pre-defined time constraints may be adjusted by the operator (e.g., via the user input device 144) in response to abnormal ECG activities such as fibrillation. In some examples, surface ECGs may be obtained via electrodes placed on a skin of the patient (not shown). In such examples, the intramyocardial electrogram signal may be optionally indexed using the surface ECGs.

In some examples, the depth navigation algorithm 128 may include a machine or deep learning-based or logic-based classification algorithm, also referred to herein as a classifier. The classification algorithm may classify the intramyocardial electrogram signal into individual depth categories based on characteristic features extracted by the feature extractor. Examples of the depth categories will be described below with respect to FIGS. 2 and 4, and example distinguishing electrogram traits of each depth category will be described with respect to FIG. 9. For example, the classification algorithm may receive the processed intramyocardial electrogram signal, such as the indexed sliding window segments and/or the segmented heartbeat cycles, as an input and output the depth category. The classification algorithm may also receive the extracted features associated with each segment as an input. The classification will be further described below with respect to FIGS. 3 and 4. Further, the depth navigation algorithm 128 may output a position-based indication and/or a category-based depth indication based on the determined depth category in order to inform the operator of the current radial depth of the intracardiac device (e.g., guidewire 110) during insertion.

Additionally or alternatively, the classification algorithm of the depth navigation algorithm 128 may include one or more deep learning networks comprising a plurality of weights and biases, activation functions, loss functions, gradient descent algorithms, and instructions for implementing the one or more deep learning networks to process the input intramyocardial electrogram signals. Additionally or alternatively, the depth navigation algorithm 128 may store instructions for implementing a neural network, such as a convolutional neural network, for classifying the processed intramyocardial electrogram segments based on the radial depth of the sensing electrode of the intracardiac device (e.g., of the exposed conductor 112) in the myocardium 104. The depth navigation algorithm 128 may include trained and/or untrained neural networks and may further include training routines, or parameters (e.g., weights and biases), associated with one or more neural network models stored therein.

In some examples, the classification algorithm of the depth navigation algorithm 128 may be trained via a training module 132. The training module 132 may comprise machine executable instructions for training the classification algorithm stored in the depth navigation algorithm 128 as well as a dataset of individual intramyocardial electrograms labeled by experienced cardiologists. For example, the dataset may include more than 20,000 labeled intramyocardial electrograms. Additional details regarding training will be described below with respect to FIG. 3.

In some examples, a sensor 146 may be positioned in the torque device 118. The sensor 146 may be an accelerometer, for example, that provides feedback to the signal processor and/or computing device 120 regarding movement of the guidewire 110. As will be elaborated herein with respect to FIG. 10, intramyocardial electrograms taken from a same location may evolve over time in a predictable manner due to myocyte recovery, and so the depth navigation algorithm 128 may utilize knowledge of whether or not the guidewire 110 is moving in classifying the electrograms.

The computing device 120 further includes the user input device 144 and a display device 130. The user input device 144 may comprise one or more of a touchscreen, a keyboard, a mouse, a trackpad, a motion sensing camera, or other device configured to enable a user to enter, interact with, and/or manipulate, data within the computing device 120. The display device 130 may include one or more display devices utilizing any type of display technology, such as a monitor, touchscreen, and/or projector. In some examples, the display device 130 may comprise a computer monitor and may display unprocessed and/or processed electrogram data as well as the depth indication. The display device 130 may be combined with the processor 124, the non-transitory memory 126, and/or the user input device 144 in a shared enclosure or may be a peripheral device.

It may be understood that the EDEN system 100 shown in FIG. 1 is one example embodiment, and other EDEN systems having similar components may also be possible. For example, another appropriate EDEN system may include more, fewer, or different components, such as an ablation catheter, a myocardial biopsy device, a myocardial drug delivery catheter or needle, a myocardial electrical stimulation or modulation device, or a myocardial pacing electrode. The intracardiac device may include one or more intracardiac sensing electrode(s) that may be used to obtain electrograms that may be processed as described herein to determine the radial depth of the intracardiac device.

Referring now to FIG. 2, a high-level block diagram of an EDEN algorithm 200 is shown. The EDEN algorithm 200 may be the depth navigation algorithm 128, for example, and may be implemented by the EDEN system 100 of FIG. 1. The EDEN algorithm 200 may be run continuously during insertion of an intracardiac device into a heart (e.g., during insertion of the guidewire 110 into the heart 101 of FIG. 1) such that data is analyzed in substantially real-time, as it is acquired, to provide real-time device depth navigation feedback.

A digitized intramyocardial electrogram signal 202, acquired by an intracardiac electrode of the intracardiac device (e.g., the exposed conductor 112 of FIG. 1), is input into a signal processor 204. The signal processor 204 may apply various digital filters, such as those described above with respect to FIG. 1. The signal processor 204 may further normalize the digitized intramyocardial electrogram signal 202, segment the intramyocardial electrogram signal 202, and/or apply other transformations.

The signal processor 204 outputs processed intramyocardial electrogram data 206, which is input into a feature extractor 208. The feature extractor 208 extracts signal features of the intramyocardial electrogram data, such as R-peaks and ST segment maximums, and outputs intramyocardial electrogram data labeled with extracted features 210. The feature extractor 208 may index the extracted features with a time stamp and/or sample number. The extracted features 210 may further include features that describe the signal and feature extraction quality, such as signal peak, noise level, and SNR. Additional details regarding the extracted features 210 are described above with respect to FIG. 1 and elaborated below with respect to FIGS. 5-8.

The extracted features 210 are applied to the processed intramyocardial electrogram data 206 to generate electrogram data with extracted features 211, and the electrogram data with extracted features 211 are input into a trained intramyocardial electrogram classifier 212. The trained intramyocardial electrogram classifier 212 may be a machine or deep learning-based classifier or logic-based classifier that is trained with ground truth labeled intramyocardial electrograms and their extracted features, as will be described below with respect to FIG. 3. In some examples, the trained intramyocardial electrogram classifier 212 includes a k-nearest neighbors classification model. Additionally or alternatively, the trained intramyocardial electrogram classifier 212 may include a support-vector machine model using a continuous wavelet transform. In still other examples, the trained intramyocardial electrogram classifier 212 may include other machine or deep learning-based or logic-based classification algorithms, including deep learning algorithms and neural networks, trained with pre-defined waveform characteristics in electrograms obtained at various myocardial depths.

The trained intramyocardial electrogram classifier 212 analyzes the input electrogram data with extracted features 211 and outputs a radial depth category 214. The radial depth category 214 may correspond to a relative depth of the electrode. The relative depth may be reported according to a pre-determined number of depth categories (e.g., radial depth positions). For example, the radial depth may be classified among five depth categories comprising intra-cameral, sub-endocardial, mid-myocardial, sub-epicardial, and extra-adventitial (e.g., intra-epicardial). As another example, the radial depth may be classified among three depth categories, with a first category comprising mid-myocardium, a second depth category comprising both sub-epicardium and sub-endocardium, and a third depth category comprising both extra-adventitial and intra-cameral. As one example, a user may select whether the output radial depth category 214 is classified according to the five depth categories or the three depth categories.

The output radial depth category 214 may be used to generate and output a real-time depth notification 216 (also referred to as a depth indication). The real-time depth notification 216 may include one or both of a position-based depth indicator 218 and a category-based depth indicator 220. The position-based depth indicator 218 may indicate the relative position of the electrode in the myocardium, such as “mid-myocardial.” The category-based depth indicator 220 may indicate a desirability of the radial depth category 214, such as according to a visual color scheme or via a text-based message. As an example, it may be most desirable to navigate the guidewire deep within the myocardium during a MIRTH procedure, making the mid-myocardium position the most desirable and depth positions outside of the myocardium (e.g., extra-adventitial and intra-cameral positions) undesirable. As such, a mid-myocardial radial depth position may be indicated by a green notification or a “desired radial position” text-based notification. As another example, a sub-epicardial and sub-endocardial depth position may be indicated by a yellow notification or a “less desirable radial position” text-based notification. As yet another example, an extra-adventitial and intra-cameral depth position may be indicated by a red notification or an “undesired radial position” text-based notification. Outputting the category-based depth indicator 220 may provide fast, uncomplicated feedback to the operator performing the device insertion, while outputting the position-based depth indicator 218 may provide additional information about device steering to remain within the myocardium.

Next, FIG. 3 shows an example of a training overview 300 that may be used to train the trained intramyocardial electrogram classifier 212 of FIG. 2. As such, components of FIG. 3 previously introduced in FIG. 2 are numbered the same and will not be re-introduced. Further, it may be understood that the training overview 300 is provided by way of example, and other training schemes may be used without departing from the scope of this disclosure.

Training electrogram data with extracted features 302 may be generated as described above for the electrogram data with extracted features 211. The training electrogram data with extracted features 302 may comprise over 20,000 individual electrogram waveforms, for example, obtained with unipolar electrodes, bipolar electrodes, multipolar electrodes, or combinations thereof. The training electrogram data with extracted features 302 receives ground truth labeling 304 regarding the radial depth position at which each waveform was acquired to generate labeled electrogram data 306. The labeled electrogram data 306 is divided into a training dataset 308 and a validation dataset 310. For example, the training dataset 308 may comprise a first portion of the labeled electrogram data 306, and the validation dataset 310 may comprise a second, remaining portion of the labeled electrogram data 306. The first portion may include more, less, or the same amount of the labeled electrogram data 306 as the second portion. The training dataset 308 is input into an untrained intramyocardial electrogram classifier 312. Based on the ground truth labels and the extracted waveform features of the labeled electrogram data 306 in the training dataset 308, the untrained intramyocardial electrogram classifier 312 learns which features have a statistical association with each radial depth position and becomes the trained intramyocardial electrogram classifier 212. The trained intramyocardial electrogram classifier 212 receives the validation dataset 310 and outputs the radial depth category 214 for each waveform of the validation dataset 310. An accuracy of the trained intramyocardial electrogram classifier 212 may be assessed based on the output radial depth category 214 relative to the ground truth labeling 304 of the validation dataset 310.

In some examples, the training electrogram data may be generated via a myocardial plunge-electrode experiment. Turning now to FIG. 4, an electrogram characterization overview 400 of unipolar electrode data from a myocardial plunge experiment relative to radial depth is shown. The electrogram characterization overview 400 includes a depth measurement 402, surface ECGs 404, intracardiac electrograms 406, relative depth indicators 418, and an axial depth schematic 420. The axial depth schematic 420 shows a short-axis view of a ventricle, including the myocardium 104, the epicardium 103, and the endocardium 105 introduced in FIG. 1. As explained above with respect to FIG. 1, the myocardium extends between the epicardium 103 and the endocardium 105. The axial depth schematic 420 further illustrates the relative depth indicators 418 in each relative position with respect to the myocardium 104, the epicardium 103, and the endocardium 105. The relative depth indicators 418 include an extra-adventitial region 408 (represented by a light shaded fill pattern), a sub-epicardial region 410 (represented by a diagonally shaded fill pattern), a mid-myocardial region 412 (represented by a dark shaded fill pattern), a sub-endocardial region 414 (represented by a vertically shaded fill pattern), and an intracavitary region 416 (represented by a medium shaded fill pattern).

As the electrode (e.g., the exposed conductor 112 of FIG. 1) is moved from the epicardial surface (e.g., the epicardium 103) through the myocardium, the intracardiac electrograms 406 change with each infinitesimal depth change from the epicardial surface. The intracardiac electrograms 406 have extractable characteristics that may be classified based on the relative depth. For example, as will be elaborated herein, the intracardiac electrograms 406 obtained in the sub-epicardial region 410 has a dominant R wave. As another example, the intracardiac electrograms 406 obtained in the mid-myocardial region 412 have maximum elevation of the ST segment. As such, data acquired via multi-channel transmyocardial plunge electrodes may be used to train an intramyocardial electrogram classifier (e.g., the untrained intramyocardial electrogram classifier of FIG. 3).

Example intramyocardial electrogram features that may be extracted (e.g., by the feature extractor 208 of the EDEN algorithm 200 of FIG. 2) will now be described with respect to FIGS. 5-8. Turning first to FIG. 5, a first graph 500 illustrating multiple waveforms of an intramyocardial electrogram signal 502 measured by a unipolar electrode (e.g., the exposed conductor 112 of FIG. 1) at an intracavitary depth is shown. The vertical axis of the first graph 500 represents a signal amplitude in voltage (e.g., V), while the horizontal axis represents time in seconds (e.g., s), as labeled. A second graph 504 illustrates a single waveform of the intramyocardial electrogram signal 502, corresponding to a portion of the first graph 500 within a box 506. The vertical axis of the second graph 504 represents the signal amplitude in voltage, while the horizontal axis represents time in milliseconds (e.g., ms), as labeled. The second graph 504 further illustrates a plurality of feature indicators and time points of interest, including a TP interval 508 comprising a duration between 0 ms and a time t1, a P wave 510 that occurs between the time t1 and a time t2, a PQ interval 512 comprising a duration between the time t2 and a time t3, a QRS complex 514 that occurs between the time t3 and a time t4, an ST segment 516 comprising a duration between the time t4 and a time t5, and a T wave 520 that occurs between the time t5 and a time t6.

Further, a T wave amplitude 522, a P wave amplitude 524, and an S wave amplitude 526 are represented by dashed lines on the second graph 504. At the intracavitary depth, the T wave amplitude 522 is the maximum signal amplitude of the intramyocardial electrogram signal 502, and the S wave amplitude 526 is the minimum signal amplitude of the intramyocardial electrogram signal 502, as shown by a signal amplitude range 530. These characteristic signal amplitude features along with the shapes of the various waves and durations of the various intervals described above may be learned by a classifier (e.g., the trained intramyocardial electrogram classifier 212 of FIG. 2) to identify electrograms obtained at the intracavitary depth in real-time during guidewire insertion.

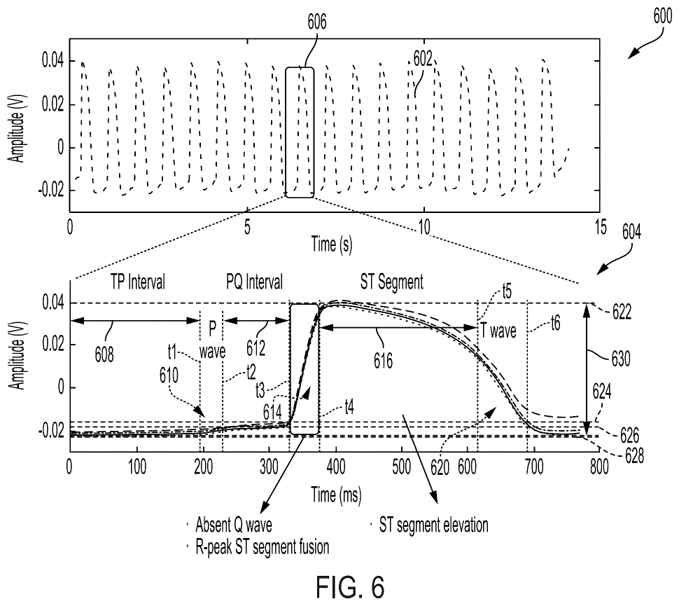

Continuing to FIG. 6, a first graph 600 illustrating multiple waveforms of an intramyocardial electrogram signal 602 measured by a unipolar electrode (e.g., the exposed conductor 112 of FIG. 1) at a mid-myocardial depth is shown. The vertical axis of the first graph 600 represents a signal amplitude in voltage (e.g., V), while the horizontal axis represents time in seconds (e.g., s), as labeled. A second graph 604 illustrates a single waveform of the intramyocardial electrogram signal 602, corresponding to a portion of the first graph 600 within a box 606. The vertical axis of the second graph 604 represents the signal amplitude in voltage, while the horizontal axis represents time in milliseconds (e.g., ms), as labeled. The second graph 604 further illustrates a plurality of feature indicators and time points of interest, including a TP interval 608 comprising a duration between 0 ms and a time t1, a P wave 610 that occurs between the time t1 and a time t2, a PQ interval 612 comprising a duration between the time t2 and a time t3, an R peak ST segment fusion 614 that occurs between the time t3 and a time t4, an ST segment 616 comprising a duration between the time t4 and a time t5, and a T wave 620 that occurs between the time t5 and a time t6.

Further, an ST segment amplitude 622, a PQ interval amplitude 624, a P wave amplitude 626, and a TP interval amplitude 628 are represented by dashed lines. The ST segment amplitude 622 is the maximum signal amplitude of the intramyocardial electrogram signal 602, and the TP interval amplitude 628 is the minimum signal amplitude of the intramyocardial electrogram signal 602, as shown by a signal amplitude range 630. These characteristic signal amplitude features along with the shapes of the various waves and durations of the various intervals described above may be learned by a classifier (e.g., the trained intramyocardial electrogram classifier 212 of FIG. 2) to identify electrograms obtained at the mid-myocardial depth in real-time during guidewire insertion. For example, the indistinguishable or R wave dominant QRS complex-ST segment fusion in combination with the maximally elevated ST segment may be used by the classifier to identify mid-myocardial electrograms.

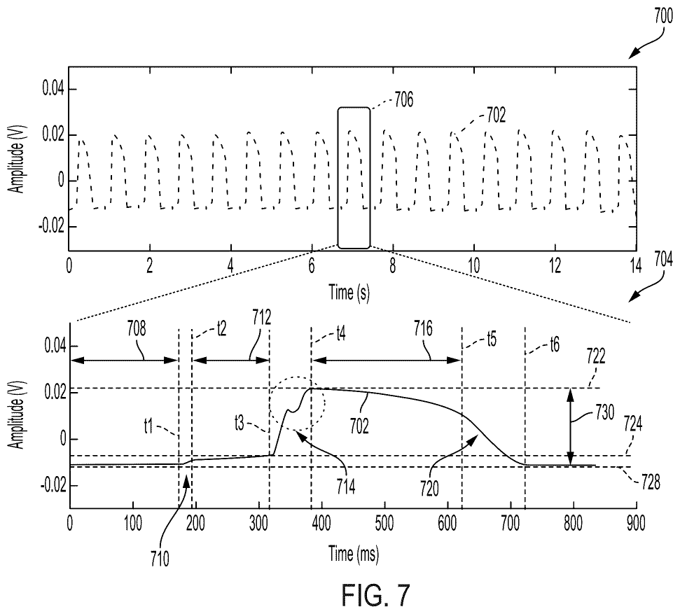

Referring now to FIG. 7, a first graph 700 illustrating multiple waveforms of an intramyocardial electrogram signal 702 measured by a unipolar electrode (e.g., the exposed conductor 112 of FIG. 1) at a sub-epicardial depth is shown. The vertical axis of the first graph 700 represents a signal amplitude in voltage (e.g., V), while the horizontal axis represents time in seconds (e.g., s), as labeled. A second graph 704 illustrates a single waveform of the intramyocardial electrogram signal 702, corresponding to a portion of the first graph 700 within a box 706. The vertical axis of the second graph 704 represents the signal amplitude in voltage, while the horizontal axis represents time in milliseconds (e.g., ms), as labeled. The second graph 704 further illustrates a plurality of feature indicators and time points of interest, including a TP interval 708 comprising a duration between 0 ms and a time t1, a P wave 710 that occurs between the time t1 and a time t2, a PQ interval 712 comprising a duration between the time t2 and a time t3, an R wave ST segment deviation 714 that occurs between the time t3 and a time t4, an ST segment 716 comprising a duration between the time t4 and a time t5, and a T wave 720 that occurs between the time t5 and a time t6.

Further, an ST segment amplitude 722, a PQ interval amplitude 724, and a TP interval amplitude 728 are represented by dashed lines. The ST segment amplitude 722 is the maximum signal amplitude of the intramyocardial electrogram signal 702, and the TP interval amplitude 728 is the minimum signal amplitude of the intramyocardial electrogram signal 702, as shown by a signal amplitude range 730. These characteristic signal amplitude features along with the shapes of the various waves and durations of the various intervals described above may be learned by a classifier (e.g., the trained intramyocardial electrogram classifier 212 of FIG. 2) to identify electrograms obtained at the sub-epicardial depth in real-time during guidewire insertion. For example, the R wave ST segment deviation in combination with the elevated ST segment may be used by the classifier to identify sub-epicardial electrograms. As another example, the classifier may distinguish sub-epicardial electrograms from mid-myocardial electrograms, such as those shown in FIG. 6, in part based on the lower ST segment amplitude 722 and the smaller signal amplitude range 730.

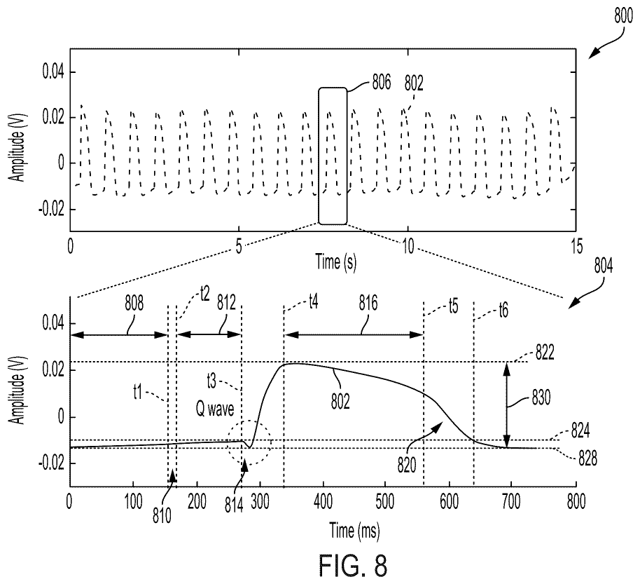

Continuing to FIG. 8, a first graph 800 illustrating multiple waveforms of an intramyocardial electrogram signal 802 measured by a unipolar electrode (e.g., the exposed conductor 112 of FIG. 1) at a sub-endocardial depth is shown. The vertical axis of the first graph 800 represents a signal amplitude in voltage (e.g., V), while the horizontal axis represents time in seconds (e.g., s), as labeled. A second graph 804 illustrates a single waveform of the intramyocardial electrogram signal 802, corresponding to a portion of the first graph 800 within a box 806. The vertical axis of the second graph 804 represents the signal amplitude in voltage, while the horizontal axis represents time in milliseconds (e.g., ms), as labeled. The second graph 804 further illustrates a plurality of feature indicators and time points of interest, including a TP interval 808 comprising a duration between 0 ms and a time t1, a P wave 810 that occurs between the time t1 and a time t2, a PQ interval 812 comprising a duration between the time t2 and a time t3, a Q wave ST segment deviation 814 that occurs between the time t3 and a time t4, an ST segment 816 comprising a duration between the time t4 and a time t5, and a T wave 820 that occurs between the time t5 and a time t6.

Further, an ST segment amplitude 822, a PQ interval amplitude 824, and a TP interval amplitude 828 are represented by dashed lines. The ST segment amplitude 822 is the maximum signal amplitude of the intramyocardial electrogram signal 802, and the TP interval amplitude 828 is the minimum signal amplitude of the intramyocardial electrogram signal 802, as shown by a signal amplitude range 830. These characteristic signal amplitude features along with the shapes of the various waves and durations of the various intervals described above may be learned by a classifier (e.g., the trained intramyocardial electrogram classifier 212 of FIG. 2) to identify electrograms obtained at the sub-endocardial depth in real-time during intracardiac device insertion. For example, the Q wave dominant ST segment deviation in combination with the elevated ST segment may be used by the classifier to identify sub-endocardial electrograms. As another example, the classifier may distinguish sub-endocardial electrograms from sub-epicardial electrograms, such as those shown in FIG. 7, in part based on the Q wave deviation 814 of the sub-endocardial electrogram. As still another example, the classifier may distinguish sub-endocardial electrograms from mid-myocardial electrograms, such as those shown in FIG. 6, based on the Q wave deviation 814 of the sub-endocardial electrogram, the lower ST segment amplitude 822, and the smaller signal amplitude range 830.

The exemplary qualitative features (e.g., extracted features) described above and other features of intramyocardial electrograms obtained at different radial depth categories are summarized in a table 900 shown in FIG. 9. A first column 902 defines a radial position, a second column 904 defines a TP segment feature, a third column 906 defines a QRS complex feature, and a fourth column 908 defines a ST segment feature for the corresponding radial position given in each row. A first row 910 summarizes features of intramyocardial electrograms obtained at intracavitary depth positions, a second row 912 summarizes features of intramyocardial electrograms obtained at sub-endocardial depth positions, a third row 914 summarizes features of intramyocardial electrograms obtained at mid-myocardial depth positions, a fourth row 916 summarizes features of intramyocardial electrograms obtained at sub-epicardial depth positions, and a fifth row 918 summarizes features of intramyocardial electrograms obtained at extra-adventitial depth positions.

As shown in the first row 910, intramyocardial electrograms obtained at intracavitary depth positions include an isoelectric TP segment (the second column 904), a QS wave (the third column 906), and an isoelectric ST segment (the fourth column 908). As shown in the second row 912, intramyocardial electrograms obtained at sub-endocardial depth positions include mild depression of the TP segment (the second column 904), a dominant Q wave (the third column 906), and mild elevation of the ST segment (the fourth column 908). As shown in the third row 914, intramyocardial electrograms obtained at mid-myocardial depth positions include maximum depression of the TP segment (the second column 904), a dominant R wave or indistinguishable QRS complex (the third column 906), and maximum elevation of the ST segment (the fourth column 908). As shown in the fourth row 916, intramyocardial electrograms obtained at sub-epicardial depth positions include mild depression of the TP segment (the second column 904), a dominant R wave (the third column 906), and mild elevation of the ST segment (the fourth column 908). As shown in the first row 910, intramyocardial electrograms obtained at extra-adventitial depth positions include an isoelectric TP segment (the second column 904), a dominant R wave (the third column 906), and an isoelectric ST segment (the fourth column 908).

Although the intracavitary (e.g., the first row 910) and extra-adventitial (e.g., the fifth row 918) qualitative features are the same for both the TP segment (e.g., the second column 904) and the ST segment (e.g., the fourth column 908), they differ for the QRS complex (e.g., the third column 906). As another example, the sub-endocardial (e.g., the second row 912) and sub-epicardial (e.g., the fourth row 916) qualitative features also differ for the QRS complex (e.g., the third column 906) alone. As such, at least one feature is different for all five of the radial depth categories, enabling a trained machine or deep learning-based classifier (e.g., the trained intramyocardial electrogram classifier 212 of FIG. 2) to identify a radial depth category of an electrogram obtained at an unknown radial depth.

Further, the characteristic features summarized in the table 900 may be present even in the presence of certain conduction abnormalities, such as left bundle branch block, characteristic of cardiomyopathies, modeled using right ventricular pacing. As such, the radial depth categories of intracardial electrograms may be effectively determined via the systems and methods described herein even in diseased hearts. Additionally, the QR features listed in table 900may depend on the orientation of the depolarization wavefront through the tissue and whether the site has rapid endocardial conduction (gives rise to endocardial Q). Some of the features may also be dependent on the sampling rate and plotted time scale, like the indistinct RS wave for the mid-myocardial electrogram. The machine or deep learning-based classifier may be trained to recognize this variation and provide robust identification of the radial depth categories.

FIG. 10 shows a set of graphs 1000 illustrating a time-based changed in intracardiac electrograms recorded via a mid-myocardial electrode that is maintained in an unmoving position and individual electrogram waveforms 1002 obtained over time. The individual electrogram waveforms 1002 include a first electrogram waveform 1004 that is measured by the mid-myocardial electrode at a first, earliest time (e.g., 0 minutes), a second electrogram waveform 1006 that is measured by the mid-myocardial electrode at a second time that is later than the first time (e.g., 2.5 minutes), and a third electrogram waveform 1008 that is measured by the mid-myocardial electrode at a third, latest time (e.g., 9.5 minutes). As can be seen by comparing the first electrogram waveform 1004, the second electrogram waveform 1006, and the third electrogram waveform 1008, an amplitude of the ST segment decreases over time while TP and PQ segments increase in amplitude over time. Further, an S wave develops and deepens over time.

Characteristic time-based amplitude changes, such as those discussed above, may be tracked over time for different ECG features to generate the set of graphs 1000. The set of graphs 1000 includes a TP interval plot 1010, a PQ interval plot 1012, an ST interval plot 1014, a P-peak plot 1016, a Q-peak plot 1018, and a T-peak plot 1020. The vertical axis of each of the set of graphs 1000 represents amplitude (e.g., in millivolts, mV), while the horizontal axis represents time (e.g., in minutes). The TP interval plot 1010 illustrates that the TP interval amplitude increases over time, particularly within the first approximately 15 minutes of electrogram recording at the same mid-myocardial location. The PQ interval plot 1012 and the P-peak plot 1016 show similar trends. The Q-peak plot illustrates that the Q-peak increases slightly in amplitude during the first approximately 10 minutes before decreasing over the next approximately 20 minutes. The ST interval plot 1014 shows an approximately exponential decrease with a half-life of approximately 12 minutes.

The set of graphs 1000 may be stored in memory, such as a part of a depth navigation algorithm (e.g., the depth navigation algorithm 128 of FIG. 1 and/or the EDEN algorithm 200 of FIG. 2) so that a processor may account for time-based changes in intramyocardial electrograms during intracardiac device insertion, such as in response to the intramyocardial electrode not moving. For example, movement (or lack thereof) may be determined based on an output of a sensor, such as an accelerometer, in a guidewire torquer (e.g., the sensor 146 of FIG. 1). It may be understood intramyocardial electrograms are a local phenomenon, and small (e.g., less than 1 millimeter) position changes in the intramyocardial electrode may avoid the time-based changes that result in constant positioning of the intramyocardial electrode. As such, sub-millimeter manipulation of the intramyocardial electrode may restore the characteristic “time zero” features of the recorded intramyocardial electrograms.

FIG. 11 shows a flow-chart of an example method 1100 for providing depth navigation feedback during insertion of an intracardiac device (e.g., a guidewire or ablation catheter) into a heart. The method 1100 is described with regard to the components of the EDEN system of FIG. 1, though it may be appreciated that the method 1100 may be implemented with other systems and components without departing from the scope of the present disclosure. The method 1100 may be carried out by a processor according to instructions stored in non-transitory memory of the EDEN system, such as the processor 124 and the non-transitory memory 126 of the computing device 120 of FIG. 1. Further, the method 1100 may include execution of a depth navigation algorithm, such as the depth navigation algorithm 128 of FIG. 1 and/or the EDEN algorithm 200 of FIG. 2.

At 1102, the method 1100 includes acquiring intramyocardial electrograms (e.g., an intracardiac electrogram signal) via one or more electrodes of the intracardiac device during insertion into the myocardium (e.g., during insertion into a heart muscle or chamber). For example, the one or more electrodes may be the exposed conductor 112 of the guidewire 110 of FIG. 1 and may acquire electrograms substantially continuously. As described with respect to FIG. 1, an analog signal from the electrode(s) may be filtered, amplified, and converted to a digital signal via a signal processor (e.g., the signal processor 122 of FIG. 1) that is electrically coupled to the electrode and in electronic communication with, or integrated with, the computing device. Alternatively, the electrode(s) can be coupled opto-electrically. The computing device may receive a continuous real-time digital signal from the signal processor or sliding window segments of the digital signal in substantially real-time. For example, each of the sliding window segments may comprise 0.2 seconds of the digital signal. The digital signal may be further normalized, transformed, and concatenated into electrogram segments comprising one complete waveform (e.g., one heartbeat cycle).

At 1104, the method 1100 includes determining if a signal quality is greater than a quality threshold. The quality threshold may be a pre-programmed value or qualifier stored in the non-transitory memory that differentiates low quality signals that may confound the depth navigation feedback, or at least not provide discernable input, from higher quality signals that may include characteristic waveform features. As one example, the quality threshold may be a threshold SNR. As another example, the quality threshold may be a threshold noise level (e.g., a threshold noise floor). As such, the processor may determine the signal quality (e.g., the SNR and/or the noise level) of each electrogram segment of the digital signal.

If the signal quality is not greater than the quality threshold, the method 1100 proceeds to 1106 and includes not processing the low quality signal. For example, portions of the digital signal that do not exceed the quality threshold may be discarded from further processing and evaluation. The method 1100 may then return so that additional intramyocardial electrograms may be acquired.

If the signal quality is greater than the quality threshold, the method 1100 proceeds to 1108 and includes extracting electrogram features from each electrogram segment in real-time via a feature extractor (e.g., the feature extractor 208 of FIG. 2). For example, the electrograms may be segmented and the feature extractor may extract signal features of each electrogram segment, such as peak and segment maximums/minimums, interval durations, etc., and output electrogram data that is labeled with the extracted features. Examples of extracted features are described with respect to FIGS. 5-8. Additionally or alternatively, the feature extractor may index the extracted features with a time stamp and/or sample number of the corresponding electrogram segment.

At 1110, the method 1100 includes inputting each electrogram segment with the extracted electrogram features into a classification algorithm. As described with respect to FIG. 3, the classification algorithm (e.g., the trained intramyocardial electrogram classifier 212 of FIGS. 2 and 3) may be trained with electrograms and their extracted features that are labeled according to the radial depth category at which they were collected by experienced cardiologists. As described with respect to FIG. 2, in some examples, the classification algorithm may use a k-nearest neighbors classification model and/or a support-vector machine to analyze the input electrogram segment with the extracted electrogram features and output a radial depth category at which the electrogram segment was acquired. For example, the classification algorithm may match the input electrogram segment with the extracted electrogram features to characteristic features corresponding to each radial depth category, such as the qualitative features shown in FIG. 9 and described above.

In some examples, the method 1100 may further include identifying movement of the intracardiac device, and thus the electrode(s), or lack thereof. For example, a persistence of movement of the electrode(s) may be monitored, and a quality of the radial depth indication may be indicated based on the monitored persistence of movement. As described with respect to FIG. 10, when the electrode(s) is left stationary for a long period (e.g., minutes), the recorded electrograms experience predictable time-dependent changes. As such, the depth navigation algorithm may use characteristic time-dependent changes stored in memory and apply various transformations and/or compensations based on an amount of time that the electrode(s) has been unmoving. The movement of the electrode(s) (or lack thereof) may be determined based on noise in the electrogram signal and/or a measured change in guidewire torque (e.g., as determined based on feedback from a sensor, such as an accelerometer).

In some examples, the depth navigation algorithm may evaluate sequential waveforms (e.g., sequential electrograms) to distinguish between radial depth categories having similar characteristic features, such as sub-epicardial and sub-endocardial or intra-cameral and extra-adventitial. For example, comparing sequential waveforms may indicate which direction the sensing electrode is coming from. As an illustrative example, a sub-epicardial electrogram, and not a sub-endocardial electrogram, is expected to be measured following an extra-adventitial electrogram. In some examples, an indication of the direction of travel of the electrode (and hence the intracardiac device) may be output as part of the real-time radial depth indication explained below, wherein the direction of travel of the intracardiac device is determined by evaluating the sequential electrograms. In still further examples, sequential waveforms/electrograms of the intracardiac electrogram signal may be evaluated along with geometric positions of the electrode(s) to determine a cumulative trajectory path of the electrode(s) (e.g., a trajectory/path of the electrode(s) over time). The geometric positions of the electrode(s) may be determined based on images of the electrode(s)/intracardiac device obtained from an imaging modality, such as X-ray images. In some examples, an indication of the cumulative trajectory path of the electrode(s) (and hence intracardiac device) may be output as part of the real-time radial depth indication explained below.

At 1112, the method 1100 includes receiving the radial depth category output from the classification algorithm. As an example, the radial depth category may be one of intra-cameral, sub-endocardial, mid-myocardial, sub-epicardial, and extra-adventitial, such as when the classification algorithm is trained to distinguish between five different radial depth categories. As another example, the radial depth category may be one of mid-myocardial, sub-epicardial/endocardial (e.g., within near a border of the myocardium), and extra-adventitial/intra-cameral (e.g., outside the myocardium), such as when the classification algorithm is trained to distinguish between three different radial depth categories.

At 1114, the method 1100 includes outputting a real-time radial depth indication, also referred to as a real-time notification, of the intracardiac device radial depth based on the radial depth category output. For example, the real-time notification may be a visual notification output to a display device (e.g., the display device 130 of FIG. 1). Additionally or alternatively, the real-time notification may be an audio notification output via a speaker system and/or haptic feedback output via a touch-based system. For example, the visual notification may include a text-based message, graphic, or other visual indicator. The audio notification may include a spoken message, tone, or other audio indicator. The haptic feedback may include a vibration or other tactile feedback.

In some examples, outputting the real-time notification of the device radial depth based on the radial depth category output includes outputting a position-based notification, as optionally indicated at 1116. For example, the position-based notification may state the radial depth category (e.g., the radial depth position of the intracardiac device/electrode with respect to the epicardium, myocardium, and endocardium). Additionally or alternatively, outputting the real-time notification of the device radial depth based on the radial depth category output includes outputting a category-based notification, as optionally indicated at 1118. The category-based notification may indicate a desirability of the device radial depth position, such as “desired radial depth position” for the mid-myocardial radial depth category, “less desired radial depth position” for the sub-endocardial and sub-epicardial radial depth categories, and “undesired radial depth position” for the extra-adventitial and intra-cameral radial depth categories. Additionally or alternatively, the category-based notification may include a graphic-based notification, such as different colors or patterns to indicate the different radial depth categories. In still further examples, the radial depth indication/notification may include a direction of travel of the electrode(s), cumulative trajectory path of the electrode(s), and/or an indication that the electrode(s) are currently stationary.

The method 1100 may then return so that the real-time notification of the device radial depth may be updated as the device position changes as new intramyocardial electrograms are acquired.

In this way, an electrocardiographic radial depth navigation system may provide real-time feedback regarding a radial position of an intracardiac device such as a guidewire during cardiac procedures, filling a gap in the position information given by imaging techniques such as fluoroscopy and echocardiography. As a result, an accuracy and efficiency of the procedure may be increased while a difficulty of performing the procedure may be decreased. Further, by using a machine or deep learning-based or logic-based depth navigation algorithm, an efficiency of the training process may be increased. Further still, the depth navigation algorithm may provide efficient electrogram processing so that radial depth navigation feedback is provided in substantially real-time. For example, electrogram features of electrograms acquired by the electrode of the intracardiac device may be extracted in real-time, as the electrograms are acquired, and a radial depth position of the electrode may be classified based on the extracted electrogram features in real-time, as the electrograms as acquired. A notification of the radial depth position may be output in real-time, as the electrograms are acquired. In other words, a first electrogram (or first series of electrograms) may be processed to extract the electrogram features, classify the radial depth position, and output the notification of the radial depth position while a second electrogram (or a second series of electrograms) is being acquired.

A technical effect of providing real-time radial position feedback during insertion of an intracardiac device in myocardium of the heart is that a target myocardial position may be more easily and accurately achieved by a catheter-based cardiac implant or other intracardiac device. A further technical effect of outputting a radial depth indication with respect to myocardium of the heart based on electronic processing of an intracardiac electrogram signal acquired via an electrode of an intracardiac device is that processing efficiency of a computing device (e.g., computing device 120 executing the depth navigation algorithm) may be increased owing to reduced reliance on imaging techniques such as fluoroscopy or echocardiography. For example, when the radial depth indication is provided as described herein (e.g., based on electronic processing of the intracardiac electrogram signal), fewer images may be acquired and/or processing of acquired images may be reduced, as the feedback of the position of the intracardiac device may be provided by the intracardiac electrogram signal instead of (or in addition to) acquired images. Additionally, the duration of the procedure (e.g., insertion of the intracardiac device into the heart) may be reduced, as the accuracy of the insertion may be increased, thus preventing or reducing inappropriate placement of the intracardiac device. The reduction in the duration of the procedure may increase processing efficiency by reducing the number of images that are acquired and/or processed, for example.

The disclosure also provides support for a method, comprising: acquiring an intracardiac electrogram signal via an electrode of an intracardiac device during insertion of the intracardiac device into a muscle or chambers of a heart, and outputting a radial depth indication with respect to myocardium of the heart based on electronic processing of the intracardiac electrogram signal. In a first example of the method, outputting the indication based on electronic processing includes determining a radial depth category of a position of the intracardiac device with respect to the myocardium of the heart based on the intracardiac electrogram signal, including: segmenting electrograms from the intracardiac electrogram signal, and extracting waveform features of each segmented electrogram. In a second example of the method, optionally including the first example, determining the radial depth category of the position of the intracardiac device with respect to the myocardium of the heart based on the intracardiac electrogram signal is based on a classifier trained to generate an output including the radial depth category responsive to the extracted waveform features. In a third example of the method, optionally including one or both of the first and second examples, the classifier comprises a machine learning-based classifier. In a fourth example of the method, optionally including one or more or each of the first through third examples, the classifier comprises a k-nearest neighbors classification model. In a fifth example of the method, optionally including one or more or each of the first through fourth examples, the classifier comprises a deep learning-based classifier or a logic-based classifier. In a sixth example of the method, optionally including one or more or each of the first through fifth examples, the classifier is trained with radial depth labeled electrograms and their extracted waveform features. In a seventh example of the method, optionally including one or more or each of the first through sixth examples, determining the radial depth category of the position of the intracardiac device with respect to myocardium of the heart based on the intracardiac electrogram signal comprises classifying the radial depth category of the intracardiac device as one of mid-myocardium, sub-epicardium, sub-endocardium, intra-cameral, and extra-adventitial. In an eighth example of the method, optionally including one or more or each of the first through seventh examples, determining the radial depth category of the position of the intracardiac device with respect to myocardium of the heart based on the intracardiac electrogram signal comprises classifying the radial depth category of the guidewire as one of a first depth category comprising mid-myocardial positions, a second depth category comprising sub-epicardial and sub-endocardial positions, and a third depth category comprising intra-cameral and extra-adventitial positions. In a ninth example of the method, optionally including one or more or each of the first through eighth examples, outputting the radial depth indication based on the radial depth category comprises outputting at least one of a position-based depth indicator and a category-based depth indicator. In a tenth example of the method, optionally including one or more or each of the first through ninth examples, the category-based depth indicator indicates a desirability of the radial depth category of the position of the intracardiac device. In an eleventh example of the method, optionally including one or more or each of the first through tenth examples, the method further comprises: monitoring a persistence of movement of the electrode, and providing a quality of the radial depth indication based on the monitored persistence of movement. In a twelfth example of the method, optionally including one or more or each of the first through eleventh examples, the method further comprises: evaluating sequential electrograms of the intracardiac electrogram signal to determine a direction of travel of the intracardiac device. In a thirteenth example of the method, optionally including one or more or each of the first through twelfth examples, the method further comprises: filtering the intracardiac electrogram signal. In a fourteenth example of the method, optionally including one or more or each of the first through thirteenth examples, the radial depth indication includes an indication of a relative position in a single dimension between endocardial and epicardial surfaces of the heart. In a fifteenth example of the method, optionally include one or more or each of the first through fourteenth examples, the method further comprises evaluating sequential electrograms of the intracardiac electrogram signal and geometric positions of the electrode to determine a cumulative trajectory path of the electrode of the intracardiac device, the geometric positions determined based on images obtained from an imaging modality, and wherein the radial depth indication includes an indication of the cumulative trajectory path.

The disclosure also provides support for a system, comprising: an intracardiac device comprising one or more electrodes, and a computing device storing instructions in non-transitory memory that, when executed, cause the computing device to: extract electrogram features of electrograms acquired by the one or more electrodes in real-time, as the electrograms are acquired, classify a radial depth position of the one or more electrodes based on the extracted electrogram features in real-time, as the electrograms are acquired, and output a notification of the radial depth position in real-time, as the electrograms are acquired. In a first example of the system, the intracardiac device comprises a guidewire or a catheter. In a second example of the system, optionally including the first example, the guidewire includes an insulated region and an uninsulated region, wherein the uninsulated region includes the one or more electrodes. In a third example of the system, optionally including one or both of the first and second examples, classifying the radial depth position of the one or more electrodes based on the extracted electrogram features in real-time, as the electrograms are acquired, is based on a classifier trained to generate an output including the radial depth position responsive to the extracted electrogram features. In a fourth example of the system, optionally including one or more or each of the first through third examples, the classifier comprises a machine learning-based classifier, a deep learning-based classifier, or a logic-based classifier. In a fifth example of the system, optionally including one or more or each of the first through fourth examples, the radial depth position includes a relative position in a single dimension between endocardial and epicardial surfaces of a heart.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
acquiring an intracardiac electrogram signal via an electrode of an intracardiac device during insertion of the intracardiac device into a muscle or chambers of a heart; and
outputting a radial depth indication with respect to myocardium of the heart based on electronic processing of the intracardiac electrogram signal.

2. The method of claim 1, wherein outputting the indication based on electronic processing includes determining a radial depth category of a position of the electrode of the intracardiac device with respect to the myocardium of the heart based on the intracardiac electrogram signal, including:
segmenting electrograms from the intracardiac electrogram signal; and
extracting waveform features of each segmented electrogram.

3. The method of claim 2, wherein determining the radial depth category of the position of the electrode of the intracardiac device with respect to the myocardium of the heart based on the intracardiac electrogram signal is based on a classifier trained to generate an output including the radial depth category responsive to the extracted waveform features.

4. The method of claim 3, wherein the classifier comprises a machine learning-based classifier or a logic-based classifier.

5. The method of claim 3, wherein the classifier comprises a k-nearest neighbors classification model.

6. The method of claim 3, wherein the classifier is trained with radial depth labeled electrograms and their extracted waveform features.

7. The method of claim 2, wherein determining the radial depth category of the position of the electrode of the intracardiac device with respect to myocardium of the heart based on the intracardiac electrogram signal comprises classifying the radial depth category of the electrode of the intracardiac device as one of mid-myocardium, sub-epicardium, sub-endocardium, intra-cameral, and extra-adventitial.

8. The method of claim 2, wherein determining the radial depth category of the position of the electrode of the intracardiac device with respect to myocardium of the heart based on the intracardiac electrogram signal comprises classifying the radial depth category of the electrode of the intracardiac device as one of a first depth category comprising mid-myocardial positions, a second depth category comprising sub-epicardial and sub-endocardial positions, and a third depth category comprising intra-cameral and extra-adventitial positions.

9. The method of claim 2, wherein outputting the radial depth indication based on the radial depth category comprises outputting at least one of a position-based depth indicator and a category-based depth indicator

10. The method of claim 9, wherein the category-based depth indicator indicates a desirability of the radial depth category of the position of the electrode of the intracardiac device.

11. The method of claim 1, further comprising monitoring a persistence of movement of the electrode, and providing a quality of the radial depth indication based on the monitored persistence of movement.

12. The method of claim 1, further comprising filtering the intracardiac electrogram signal and/or evaluating sequential electrograms of the intracardiac electrogram signal to determine a direction of travel of the electrode of the intracardiac device.

13. The method of claim 1, wherein the radial depth indication includes an indication of a relative position of the electrode in a single dimension between endocardial and epicardial surfaces of the heart.

14. The method of claim 1, further comprising evaluating sequential electrograms of the intracardiac electrogram signal and geometric positions of the electrode to determine a cumulative trajectory path of the electrode of the intracardiac device, the geometric positions determined based on images obtained from an imaging modality, and wherein the radial depth indication includes an indication of the cumulative trajectory path.

15. A system, comprising:
an intracardiac device comprising one or more electrodes; and
a computing device storing instructions in non-transitory memory that, when executed, cause the computing device to:
extract electrogram features of electrograms acquired by the one or more electrodes in real-time, as the electrograms are acquired;
classify a radial depth position of the one or more electrodes based on the extracted electrogram features in real-time, as the electrograms are acquired; and
output a notification of the radial depth position in real-time, as the electrograms are acquired.

16. The system of claim 15, wherein the intracardiac device comprises a guidewire or a catheter, and wherein the one or more electrodes include a unipolar electrode or a bipolar electrode.

17. The system of claim 16, wherein the guidewire includes an insulated region and an uninsulated region, wherein the uninsulated region includes the one or more electrodes.

18. The system of claim 15, wherein classifying the radial depth position of the one or more electrodes based on the extracted electrogram features in real-time, as the electrograms are acquired, is based on a classifier trained to generate an output including the radial depth position responsive to the extracted electrogram features.

19. The system of claim 18, wherein the classifier comprises a machine learning-based classifier, a deep learning-based classifier, or a logic-based classifier.

20. The system of claim 15, wherein the radial depth position includes a relative position in a single dimension between endocardial and epicardial surfaces of a heart.
